(19) United States
(12) Patent Application Publication
(10) Pub. No.: US 2024/0009117 A1
(43) Pub. Date: Jan. 11, 2024

(54) ENHANCED UPTAKE TRANSMUCOSAL DRUG DELIVERY COMPOSITIONS AND METHODS OF USE

(71) Applicant: Padagis Israel Pharmaceuticals Ltd., Allegan, MI (US)

(72) Inventors: Shai ABUTBUL, Shoham (IL); Smadar ATSMON, Shoham (IL)

(21) Appl. No.: 18/252,012

(22) PCT Filed: Nov. 2, 2021
(86) PCT No.: PCT/US2021/057697

§ 371 Date: May 5, 2023

Related U.S. Application Data
(60) Provisional application No. 63/110,232, filed on Nov. 5, 2020.

(51) Int. Cl. A61K 9/00; A61K 47/18; A61K 47/10; A61K 31/4045

(52) U.S. Cl. CPC A61K 9/0043; A61K 47/186; A61K 47/10; A61K 31/4045

(57) ABSTRACT

Compositions for transmucosal administration of active pharmaceutical ingredient (API) are disclosed. More specifically, the formulations of the present invention provide an intranasal administration which can exhibit a rapid high plasma concentration. The formulations of the present invention comprise an API and penetration enhancing agents which facilitate the penetration of said API through the mucosa. Methods for improved treatment of conditions by administration of said formulations having enhanced penetration via the mucosa are also disclosed.

### CROSS-REFERENCE TO RELATED APPLICATION

This application claims the benefit of U.S. Provisional Application No. 63/110,232, filed Nov. 5, 2020, the disclosure of which is hereby expressly incorporated by reference herein in its entirety.

### FIELD OF THE INVENTION

The present invention relates to compositions for nasal administration in which the penetration via the nasal membrane is enhanced. More specifically, the formulations of the present invention provide a nasal administration which exhibits a rapid high plasma concentration.

### BACKGROUND OF THE INVENTION

Certain biologically active compounds are better absorbed through the mucosa than through other routes of administration, such as through the stomach or intestine. Transmucosal absorption allows permeation of drugs directly into the bloodstream and then into the cells within a matter of minutes. More particularly, intranasal administration generally allows the active agent to bypass first-pass metabolism, thereby enhancing the bioavailability of the active agent. Such delivery can offer several advantages over other modes of drug delivery including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Intranasal administration of a drug also can facilitate compliance, especially for pediatric patients, geriatric patients, patients suffering from a neurodegenerative disease, or other patients for which swallowing is difficult, e.g., patients suffering from nausea such as patients undergoing chemotherapy, or patients with a swallowing disorder. There continues to be a need for compositions that achieve improved uptake of pharmaceutically effective amounts of active pharmaceutical ingredients (API) when administered intranasally.

### SUMMARY OF THE INVENTION

One aspect of the present disclosure encompasses a pharmaceutical composition for intranasal administration of an active pharmaceutical ingredient (API). The composition comprises the API and penetration enhancing agents. The penetration enhancing agent comprise benzalkonium chloride and propylene glycol. The intranasal administration can be administered as a spray, drops, an aerosol, a paste, a cream, a gel, an ointment, a salve, a foam, a lotion, an oil suspension, an emulsion, or a solution.

The penetration enhancing agents can comprise benzalkonium chloride at a concentration ranging from about 0.05 to about 0.5% w/w. The penetration enhancing agents comprise propylene glycol at a concentration ranging from about 1.5 to about 12% w/w. In some aspects, the penetration enhancing agents comprise benzalkonium chloride at a concentration ranging from about 0.05 to about 0.5% w/w, and propylene glycol at a concentration ranging from about 1.5 to about 12% w/w. The composition can further comprise a pH modifier.

The API can be Naloxone, zolmitriptan, midazolam, diazepam, epinephrine, esketamin, painkillers, sumatriptan, nitroglycerin, or combinations thereof. In some aspects, the API is sumatriptan. When the API is sumatriptan, the sumatriptan can be at a concentration ranging from about 9 to about 11% w/w of the composition. Administration of the composition comprising sumatriptan can provide a Cmax ranging from about 1.5 time to about 3 times higher than the Cmax obtained upon administration of a composition comprising sumatriptan and BZK without PG.

Another aspect of the present disclosure encompasses a method of intranasal administration of a pharmaceutically active ingredient (API) to a subject in need thereof. The method comprises administering to the subject through an intranasal administration route a composition comprising the API and penetration enhancing agents. The agents comprise benzalkonium chloride and propylene glycol. Intranasal administration can be administered as a spray, drops, an aerosol, a paste, a cream, a gel, an ointment, a salve, a foam, a lotion, an oil suspension, an emulsion, or a solution. In some aspects, transmucosal administration is administered as a nasal spray or a nasal drop.

The penetration enhancing agents can comprise benzalkonium chloride at a concentration ranging from about 0.05 to about 0.5% w/w. The penetration enhancing agents comprise propylene glycol at a concentration ranging from about 1.5 to about 12% w/w. In some aspects, the penetration enhancing agents comprise benzalkonium chloride at a concentration ranging from about 0.05 to about 0.5% w/w, and propylene glycol at a concentration ranging from about 1.5 to about 12% w/w. The composition can further comprise a pH modifier.

The API can be Naloxone, zolmitriptan, midazolam, diazepam, epinephrine, esketamin, painkillers, sumatriptan, nitroglycerin, or combinations thereof. In some aspects, the API is sumatriptan. When the API is sumatriptan, the sumatriptan can be at a concentration ranging from about 9 to about 11% w/w of the composition. Administration of the composition comprising sumatriptan can provide a Cmax ranging from about 1.5 time to about 3 times higher than the Cmax obtained upon administration of a composition comprising sumatriptan and BZK without PG.

In an additional aspect, the present disclosure encompasses a pharmaceutical composition for intranasal administration of sumatriptan. The composition comprises about 9 to about 11% w/w sumatriptan. The composition also comprises penetration enhancing agents. The penetration enhancing agents comprise about 0.05 to about 0.5% w/w benzalkonium chloride and about 1.5 to about 12% w/w propylene glycol.

In yet another aspect, the present disclosure encompasses a method of intranasal administration of sumatriptan to a subject in need thereof. The method comprises administering to the subject through an intranasal administration route a composition comprising about 9 to about 11% w/w sumatriptan. The composition also comprises penetration enhancing agents. The penetration enhancing agents comprise about 0.05 to about 0.5% w/w benzalkonium chloride and about 1.5 to about 12% w/w propylene glycol.

In another aspect, the present disclosure encompasses a method of treating pain in a subject in need thereof. The method comprises intranasally administering to the subject a composition comprising a painkiller and penetration enhancing agents. The pain medication can be sumatriptan. The penetration enhancing agents comprise benzalkonium chloride and propylene glycol.

The benzalkonium chloride can be at a concentration ranging from about to about 0.5% w/w, and the propylene glycol can be at a concentration ranging from about 1.5 to about 12% w/w. In some aspects, the penetration enhancing agents comprise benzalkonium chloride at a concentration ranging from about 0.05 to about w/w, and propylene glycol at a concentration ranging from about 1.5 to about 12% w/w.

The pain can be migraine pain. The painkiller can be a migraine medication. The painkiller can be sumatriptan. The sumatriptan can be at a concentration ranging from about 9 to about 11% w/w of the composition, and the penetration enhancing agents can comprise benzalkonium chloride at a concentration ranging from about 0.05 to about w/w and propylene glycol at a concentration ranging from about 1.5 to about 12% w/w. When the painkiller is sumatriptan, administration of the composition can provide a Cmax ranging from about 1.5 time to about 3 times higher than the Cmax obtained upon administration of a composition comprising sumatriptan and BZK without PG.

In yet an additional aspect, the present disclosure encompasses a method of treating pain in a subject in need thereof. The method comprises intranasally administering to the subject a composition comprising a painkiller and penetration enhancing agents. The agents comprise about 0.05 to about 0.5% w/w benzalkonium chloride and from about 1.5 to about 12% w/w propylene glycol. The painkiller can be sumatriptan. When the painkiller is sumatriptan, the sumatriptan can be at a concentration ranging from about 9 to about 11% w/w of the composition.

In another aspect, the present disclosure encompasses a method of treating a migraine in a subject in need thereof. The method comprises intranasally administering a composition comprising a migraine medication to the subject. The composition comprises a migraine medication and penetration enhancing agents. The agents comprise benzalkonium chloride and propylene glycol. The benzalkonium chloride can be at a concentration ranging from about 0.05 to about 0.5% w/w, and the propylene glycol can be at a concentration ranging from about 1.5 to about 12% w/w. In some aspects, the penetration enhancing agents comprise benzalkonium chloride at a concentration ranging from about 0.05 to about 0.5% w/w, and propylene glycol at a concentration ranging from about 1.5 to about 12% w/w.

The migraine medication can be sumatriptan. When the migraine medication is sumatriptan, sumatriptan can be at a concentration ranging from about 9 to about 11% w/w of the composition. Further, when the migraine medication is sumatriptan, the penetration enhancing agents can comprise benzalkonium chloride at a concentration ranging from about 0.05 to about 0.5% w/w, and propylene glycol at a concentration ranging from about 1.5 to about 12% w/w. Administration of the composition can provide a Cmax ranging from about 1.5 time to about 3 times higher than the Cmax obtained upon administration of a composition comprising sumatriptan and BZK without PG.

In one aspect, the present disclosure encompasses a method of treating migraine pain in a subject in need thereof. The method comprises intranasally administering to the subject a composition comprising sumatriptan at a concentration of about 9 to about 11% w/w sumatriptan and penetration enhancing agents. The agents comprise about 0.05 to about 0.5% w/w benzalkonium chloride and about 1.5 to about 12% w/w propylene glycol.

Yet another aspect of the present disclosure encompasses a pharmaceutical kit comprising the pharmaceutical composition described above for intranasal administration of an active pharmaceutical ingredient (API).

### BRIEF DESCRIPTION OF THE FIGURES

FIG. 1 depicts a graph of the plasma concentration of sumatriptan over time.

FIG. 2 depicts a graph of the plasma concentration of sumatriptan over time.

### DETAILED DESCRIPTION

The present disclosure encompasses compositions for transmucosal administration of an active compound and methods of using the compositions for providing immediate uptake of a pharmaceutically effective amount of the API. Compositions that achieve improved uptake of the API by using two or more penetration enhancing agents comprising a combination of benzalkonium chloride and propylene glycol were discovered. More specifically, the inventors discovered that a combination of propylene glycol and benzalkonium chloride have a significant impact on the pharmacokinetic profile of APIs, significantly increasing the uptake of the API at substantially shorter periods of time when compared to administration of the API in the absence of benzalkonium chloride and propylene glycol.

The formulations of the present invention further relate to improved treatment of conditions by administration of said compositions having enhanced penetration via the mucosa. The compositions of the invention permit efficacious non-invasive delivery of APIs directly into the general systemic circulation or into the central nervous system through the nasal mucosa. Such route of delivery eliminates a need for large drug doses and drug deactivation connected with oral drug delivery, or invasive routes of drug administration, such as intravenous, intramuscular, intraperitoneal, cutaneous or subcutaneous injections requiring visits to the doctor's office and/or assistance of medical personnel. The delivery of the APIs according to the disclosure bypasses the gastrointestinal tract absorption, liver metabolism and kidney deactivation, and delivers the drug directly to the systemic blood circulation.

### I. Composition

One aspect of the present disclosure encompasses a pharmaceutical composition for transmucosal administration of an API. In some aspects, transmucosal delivery is intranasal delivery. The composition comprises the API and two or more penetration enhancing agents. The API, the two or more penetration enhancing agents, and other components that can be used in a composition of the disclosure are described below.

(a) Active Pharmaceutical Ingredient (API)

Suitable APIs include, without limit, opioid analgesic agents (e.g., adulmine, alfentanil, allocryptopine, allylprodine, alphaprodine, anileridine, aporphine, benzylmorphine, berberine, bicuculine, bicucine, bezitramide, buprenorphine, bulbocaprine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, le-vorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, nalmefene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tapentadol, tilidine, and tramadol); opioid antagonists (e.g., naloxone, naltrexone, alvimopan, cyprodime, diprenorphine, gemazocine, 5′-guanidinonaltrindole, levallorphan, methylnaltrexone, naldemedine, nalmexone, nalorphine, naloxazone, naloxol, naloxonazine, 6β-naltrexol-d4, naltriben, naltrindole, norbinaltorphimine, oxilorphan, quadazocine, and samidorphan); non-opioid analgesic agents (e.g., acetylsalicylic acid, acetaminophen, paracetamol, ibuprofen, ketoprofen, indomethacin, diflunisol, naproxen, ketorolac, dichlophenac, tolmetin, sulindac, phenacetin, piroxicam, and mefamanic acid); anti-inflammatory agents (e.g., glucocorticoids such as alclometasone, fluocinonide, methylprednisolone, triamcinolone and dexamethasone; non-steroidal anti-inflammatory agents such as celecoxib, deracoxib, ketoprofen, lumiracoxib, meloxicam, parecoxib, rofecoxib, and valdecoxib); antitussive agents (e.g., dextromethorphan, codeine, hydrocodone, caramiphen, carbetapentane, and dextromethorphan); antipyretic agents (e.g., acetylsalicylic acid and acetaminophen); antibiotic agents (e.g., aminoglycosides such as, amikacin, gentamicin, kanamycin, neomycin, netilmicin, streptomycin, and tobramycin; carbecephem such as loracarbef; carbapenems such as certapenem, imipenem, and meropenem; cephalosporins such as cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone; macrolides such as azithromycin, clarithromycin, dirithromycin, erythromycin, and troleandomycin; monobactam; penicillins such as amoxicillin, ampicillin, carbenicillin, cloxacillin, dicloxacillin, nafcillin, oxacillin, penicillin G, penicillin V, piperacillin, and ticarcillin; polypeptides such as bacitracin, colistin, and polymyxin B; quinolones such as ciprofloxacin, enoxacin, gatifloxacin, levofloxacin, lomefloxacin, moxifloxacin, norfloxacin, ofloxacin, and trovafloxacin; sulfonamides such as mafenide, sulfacetamide, sulfamethizole, sulfasalazine, sulfisoxazole, and trimethoprim sulfamethoxazole; tetracyclines such as demeclocycline, doxycycline, minocycline, and oxytetracycline); anti-microbial agents (e.g., ketoconazole, amoxicillin, cephalexin, miconazole, econazole, acyclovir, and nelfinavir); antiviral agents (e.g., acyclovir, gangciclovir, oseltamivir, and relenza); steroids (e.g., estradiol, testosterone, cortisol, aldosterone, prednisone, and cortisone); amphetamine stimulant agents (e.g., amphetamine and amphetamine-like drugs); non-amphetamine stimulant agents (e.g., methylphenidate, nicotine, and caffeine); laxative agents (e.g., bisacodyl, casanthranol, senna, and castor oil); anti-nausea agents (e.g., dolasetron, granisetron, ondansetron, tropisetron, meclizine, and cyclizine); anorexic agents (e.g., fenfluramine, dexfenfluramine, mazindol, phentermine, and aminorex); antihistaminic agents (e.g., phencarol, cetirizine, cinnarizine, ethamidindole, azatadine, brompheniramine, hydroxyzine, and chlorpheniramine); antiasthmatic agents (e.g., zileuton, montelukast, omalizumab, fluticasone, and zafirlukast); antidiuretic agents (e.g., desmopressin, vasopressin, and lypressin); antimigraine agents (e.g., naratriptan, frovatriptan, eletriptan, dihydroergotamine, zolmitriptan, almotriptan, and sumatriptan); antispasmodic agents (e.g., dicyclomine, hyoscyamine, and peppermint oil); antidiabetic agents (e.g., methformin, acarbose, miglitol, pioglitazone, rosiglitazone, nateglinide, repaglinide, mitiglinide, saxagliptin, sitagliptine, vildagliptin, acetohexamide, chlorpropamide, gliclazide, glimepiride, glipizide, glyburide, tolazamide, and tolbutamide); respiratory agents (e.g., albuterol, ephedrine, metaproterenol, and terbutaline); sympathomimetic agents (e.g., pseudoephedrine, phenylephrine, phenylpropanolamine, epinephrine, norepinephrine, dopamine, and ephedrine); H2 blocking agents (e.g., cimetidine, famotidine, nizatidine, and ranitidine); antihyperlipidemic agents (e.g., clofibrate, cholestyramine, colestipol, fluvastatin, atorvastatin, genfibrozil, lovastatin, niacin, pravastatin, fenofibrate, colesevelam, and simvastatin); antihypercholesterol agents (e.g., lovastatin, simvastatin, pravastatin, fluvastatin, atorvastatin, cholestyramine, colestipol, colesevelam, nicotinic acid, gemfibrozil, and ezetimibe); cardiotonic agents (e.g., digitalis, ubidecarenone, and dopamine); vasodilating agents (e.g., nitroglycerin, captopril, dihydralazine, diltiazem, and isosorbide dinitrate); vasoconstricting agents (e.g., dihydroergotoxine and dihydroergotamine); anticoagulants (e.g., warfarin, heparin, and Factor Xa inhibitors); sedative agents (e.g., amobarbital, pentobarbital, secobarbital, clomethiazole, diphenhydramine hydrochloride, and alprazolam); hypnotic agents (e.g., zaleplon, zolpidem, eszopiclone, zopiclone, chloral hydrate, and clomethiazole); anticonvulsant agents (e.g., lamitrogene, oxycarbamezine, phenytoin, mephenytoin, ethosuximide, methsuccimide, carbamazepine, valproic acid, gabapentin, topiramate, felbamate, and phenobarbital); muscle-relaxing agents (e.g., baclofen, carisoprodol, chlorzoxazone, cyclobenzaprine, dantrolene sodium, metaxalone, orphenadrine, pancuronium bromide, and tizanidine); antipsychotic agents (e.g., phenothiazine, chlorpromazine, fluphenazine, perphenazine, prochlorperazine, thioridazine, trifluoperazine, haloperidol, droperidol, pimozide, clozapine, olanzapine, risperidone, quetiapine, ziprasidone, melperone, and paliperidone); antianxiolitic agents (e.g., lorazepam, alprazolam, clonazepam, diazepam, buspirone, meprobamate, and flunitrazepam); antihyperactive agents (e.g., methylphenidate, amphetamine, and dextroamphetamine); antihypertensive agents (e.g., alphamethyldopa, chlortalidone, reserpine, syrosingopine, rescinnamine, prazosin, phentolamine, felodipine, propanolol, pindolol, labetalol, clonidine, captopril, enalapril, and lisonopril); antineoplasia agents (e.g., taxol, actinomycin, bleomycin A2, mitomycin C, daunorubicin, doxorubicin, epirubicin, idarubicin, and mitoxantrone); soporific agents (e.g., zolpidem tartrate, eszopiclone, ramelteon, and zaleplon); tranquilizer agents (e.g., alprazolam, clonazepam, diazepam, flunitrazepam, lorazepam, triazolam, chlorpromazine, fluphenazine, haloperidol, loxapine succinate, perphenazine, prochlorperazine, thiothixene, and trifluoperazine); decongestant agents (e.g., ephedrine, phenylephrine, naphazoline, and tetrahydrozoline); beta blockers (e.g., levobunolol, pindolol, timolol maleate, bisoprolol, carvedilol, and butoxamine); alpha blockers (e.g., doxazosin, prazosin, phenoxybenzamine, phentolamine, tamsulosin, alfuzosin, and terazosin); non-steroidal hormones (e.g., corticotropin, vasopressin, oxytocin, insulin, oxendolone, thyroid hormone, and adrenal hormone); erectile dysfunction improvement agents; herbal agents (e.g., glycyrrhiza, aloe, garlic, Nigella sativa, rauwolfia, St John's wort, and valerian); enzymes (e.g., lipase, protease, amylase, lactase, lysozyme, and urokinase); humoral agents (e.g., prostaglandins, natural and synthetic, for example, PGE1, PGE2alpha, PGF2alpha, and the PGE1 analog misoprostol); psychic energizers (e.g., 3-(2-aminopropy)indole and 3-(2-aminobutyl)indole); nutritional agents; essential fatty acids; non-essential fatty acids; vitamins; minerals; and combinations thereof.

Any one or a combination of APIs may be incorporated in the pharmaceutical composition described herein in any suitable form, such as, for example, as a pharmaceutically acceptable salt, uncharged or charged molecule, molecular complex, solvate or hydrate, prodrug, and, if relevant, isomer, enantiomer, racemic mixture, and/or mixtures thereof. Furthermore, the API may be in any of its crystalline, semi-crystalline, amorphous, or polymorphous forms.

In some aspects, the pharmaceutically active agent is Naloxone (opioid), zolmitriptan, midazolam, diazepam, epinephrine, esketamin, painkillers, sumatriptan, nitroglycerin, or combinations thereof.

In some aspects, the API is a painkiller. In one aspect, the painkiller is a migraine medication. The current therapies to treat migraine headaches include the administration of serotonin 5HT1B/1D receptor agonists collectively called as triptans, which chemically are tryptamine-based drugs. “Triptan” includes sumatriptan, rizatriptan, naratriptan, zolmitriptan, eletriptan, almotriptan, frovatriptan and/or a pharmaceutically acceptable salt, solvate, hydrate, polymorph, prodrug or mixtures thereof. “Salt of sumatriptan” is intended to include, but is not limited to, any of the following salts of sumatriptan: sulphate salt, citrate salt, phosphate salt, maleate salt, formate salt, and acetate salt. Other examples of salts of sumatriptan include, but are not limited to, hydrochloride, hydrobromide, nitrate, mesylate, benzoate and succinate. ‘Salt of sumatriptan’ is also intended to include above-defined salt of sumatriptan formed either externally or in situ during the preparation of the composition, by addition of the corresponding acid.

The amount of the API in the pharmaceutical composition can and will vary depending on the active agent, the method of administration, the composition, the subject, among other variables, and can be determined experimentally. When the API is sumatriptan or a salt thereof, the amount of sumatriptan in the pharmaceutical composition may range from about 1% to about 50% w/w of the pharmaceutical composition. In various aspects, the amount of sumatriptan in the pharmaceutical composition may range from about 2% to about 40% w/w, from about 3% to about 30% w/w, from about 4% w/w to about 20% w/w, or from about 5% w/w to about 25% w/w. In certain aspects, the amount of sumatriptan in the pharmaceutical composition may be about 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or about 25% w/w of the pharmaceutical composition. In some aspects, the amount of sumatriptan in the pharmaceutical composition ranges from about 9 to about 11% w/w of the pharmaceutical composition.

(b) Penetration Enhancing Agents

The pharmaceutical compositions disclosed herein comprise two or more penetration enhancing agents among other ingredients normally used in formulations. Penetration enhancers act by several mechanisms, which include increasing retention time of drug around the mucosa, interaction with the mucosal protein and intracellular lipid, and/or enhancing the drug partitioning across the mucosa.

Classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics such as macrocylic lactones, ketones, and anhydrides and cyclic ureas, surfactants, N-methyl pyrrolidones and derivatives thereof, DMSO and related compounds, ionic compounds, and related compounds, and solvents such as alcohols, ketones, amides, polyols (e.g., glycols). Examples of these classes are known in the art.

The two or more penetration enhancing agents of the instant disclosure comprise one or more benzalkonium chlorides. Benzalkonium chlorides (BACs; BZK; BKC; BAK; BAC), also known as alkyl dimethyl benzyl ammonium chlorides, alkyl dimethyl (phenylmethyl) quaternary ammonium chlorides, ammonium alkyl dimethyl (phenylmethyl) chlorides, or ammonium alkyl dimethyl benzyl chlorides, are a class of quaternary ammonium compounds (QACs), shown below. In some aspects, the two or more penetration enhancing agents of the instant disclosure comprise benzalkonium chloride.

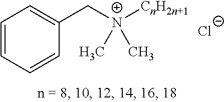

In certain aspects, the amount of BZK in the pharmaceutical composition ranges from about 0.01% w/w to about 10% w/w, about 0.03% w/w to about 1% w/w, about 0.03% w/w to about 0.7% w/w, or about 0.1% to about 0.3% w/w of the pharmaceutical composition. For instance, the amount of BZK in the pharmaceutical composition can be about 0.05, 0.06, 0.07, 0.08, 0.09, 0.1, 0.11, 0.12, 0.13, 0.14, 0.15, 0.17, 0.18, 0.19, 0.2, 0.21, 0.22, 0.23, 0.24, 0.25, 0.26, 0.27, 0.28, 0.29, 0.3, 0.31, 0.33, 0.34, 0.35, 0.36, 0.37, 0.38, 0.39, 0.4, 0.41, 0.42, 0.43, 0.44, 0.45, 0.46, 0.47, 0.49, or about 0.5% w/w of the pharmaceutical composition.

The two or more penetration enhancing agents of the composition also comprise propylene glycol (PG). In certain aspects, the amount of PG in the pharmaceutical composition ranges from about 0.5% w/w to about 25% w/w, about 1.5% w/w to about 20% w/w, about 1% w/w to about 15% w/w, about 1.5% w/w to about 12% w/w, or about 3.5% w/w to about 10% w/w of the pharmaceutical composition. For instance, the amount of PG in the pharmaceutical composition can be about 1, 1.5, 2, 2.5, 3, 3.5, 4, 4.5, 5, 5.5, 6, 6.5, 7, 7.5, 8, 8.5, 9, 9.5, 10, 10.5, 11, 11.5, or about 12% w/w of the pharmaceutical composition.

In some aspects, the penetration enhancing agents comprise BZK in an amount ranging from about 0.05-0.5% w/w of the pharmaceutical composition, and PG in an amount ranging from about 1.5-12% w/w of the pharmaceutical composition.

Upon administration of a composition comprising an API and a combination of the BZK and PG penetration enhancing agents as disclosed herein, the composition provides a substantially higher Cmax and shorter Tmax than the Cmax and Tmax obtained upon administration of a comparative composition without one or both of the penetration enhancing agents. A composition comprising an API and BZK and PG as penetration enhancers of the instant disclosure provides a Cmax ranging from about 1.2 to about 5 times higher, or about 1.5 time to about 3 times higher compared to administration of the comparative compositions. A composition comprising an API and BZK and PG as penetration enhancers of the instant disclosure provides Tmax ranging from about 2 to about 100 times shorter, about 10 times to about 80 times shorter, or about 40 to about 70 times shorter than the Tmax obtained upon administration of a comparative composition without one or both of the penetration enhancing agents.

When the API is sumatriptan, administration of a composition comprising sumatriptan and a combination of BZK and PG penetration enhancers of the instant disclosure provides a Cmax ranging from about 1.2 to about 5 times higher, or about 1.5 times to about 3 times higher than the Cmax obtained upon administration of a comparative composition comprising sumatriptan and BZK without PG.

(c) Other Components

A composition of the invention may be formulated and administered transmucosally to a subject by several different means. For instance, a composition for nasal administration may generally be administered in the form of a spray, drops, a powder, an aerosol, a paste, a cream, a gel, an ointment, a salve, a foam, a lotion, a cream, an oil suspension, an emulsion, a solution, and the like in dosage unit formulations containing conventional nontoxic pharmaceutically acceptable adjuvants, carriers, excipients, and vehicles as desired. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Formulations of various embodiments may include the composition of the disclosure, along with an excipient. Non-limiting examples of excipients include preservatives (antioxidants), flavor-modifying agents, tonicity agents such as NaCl, suspending agents, coloring agents, pH modifiers, chelating agents, antimicrobial agents, release-controlling polymers, and combinations of any of these agents.

i. Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apocarotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanillic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof.

ii. Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include, but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226, 5,075,114, and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

iii. Colorants

Depending upon the aspect, it may be desirable to include a coloring agent. Suitable color additives include, but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects.

iv. pH Modifiers

Non-limiting examples of pH modifiers include citric acid, acetic acid, tartaric acid, malic acid, fumaric acid, lactic acid, phosphoric acid, sorbic acid, benzoic acid, potassium and sodium phosphate, sodium carbonate, and sodium bicarbonate.

v. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

vi. Antimicrobial Agents

An antimicrobial agent may be included as an excipient to minimize the degradation of the compound according to this disclosure by microbial agents, including but not limited to, bacteria and fungi. Non-limiting examples of antimicrobials include para-bens, chlorobutanol, phenol, calcium propionate, sodium nitrate, sodium nitrite, Na2EDTA, and sulfites including but not limited to sulfur dioxide, sodium bisulfite, and potassium hydrogen sulfite.

### II. Methods of Use

Another aspect of the present disclosure encompasses a method of transmucosal administration of a pharmaceutically active ingredient (API) to a subject in need thereof. The method comprises administering to the subject through a transmucosal route a composition comprising the API and penetration enhancing agents. The composition, the API and the penetration enhancing agents are described above in Section I.

(a) Subject

The subject can be a human, a livestock animal, a companion animal, a lab animal, or a zoological animal. In one aspect, the subject may be a rodent, e.g., a mouse, a rat, a guinea pig, etc. Non-limiting examples of suitable livestock animals may include pigs, cows, horses, goats, sheep, llamas and alpacas. Non-limiting examples of companion animals may include pets such as dogs, cats, rabbits, and birds. As used herein, a “zoological animal” refers to an animal that may be found in a zoo. Such animals may include non-human primates, large cats, wolves, and bears. Non-limiting examples of a laboratory animal may include rodents, canines, felines, and non-human primates. Non-limiting examples of rodents may include mice, rats, guinea pigs, etc. In some aspects, the subject is a human subject.

In some aspects, the subject suffers from craniofacial pain or head pain. In some aspects, the pain is a chronic pain associated with migraine, a cluster headache, a tension headache, a secondary type of headache or trigeminal neuralgia. In some aspects, the pain is a sharp and shooting pain associated with movement. In some aspects, the pain is a neuropathic pain caused by surgery related nerve injury.

(b) Administration

The methods comprise transmucosal administration of a composition comprising the API and penetration enhancing agents to the subject through a transmucosal route. Administering a composition through a transmucosal route comprises contacting a mucosa of the subject with the composition. The mucosa may be oral, buccal, sublingual, ocular, gingival, palatal, nasal, nasopharynxal, oropharynxal, conjunctival, vaginal, and gastrointestinal mucosa, among others. Accordingly, administering a composition of the disclosure comprises oral, buccal, sublingual, ocular, gingival, palatal, nasal, nasopharynxal, oropharynxal, conjunctival, vaginal, and gastrointestinal administration.

In some aspects, the method comprises nasally administering the composition through the nasal mucosa. When administered transmucosally through the nasal mucosa, the compositions can be administered by inhalation using any spray or aerosol bottle, or by instillation using a syringe, dropper, or the like.

As used herein and unless otherwise indicated, the term “nasal mucosa” refers to nasal mucosal membranes lining the nasal cavity. Importantly, drug administration via the nasal cavity allows direct transport of drugs into the central nervous system via the pathways of olfactory and trigeminal nerve, and is likely the most rapid way to deliver drugs to the brain. Intranasal absorption features low lipophilicity, enzymatic degradation within the nasal cavity, large molecular size, and rapid mucociliary clearance from the nasal passages, which explains the low risk of systemic exposure of the administered drug absorbed via intranasal.

For therapeutic applications, a therapeutically effective amount of an API of the invention is administered to a subject. As used herein, the term “therapeutically effective amount” of an API refers to an amount of the API sufficient to produce a measurable effect on a condition. Actual dosage levels of active ingredients in a therapeutic composition of the invention may be varied so as to administer an amount of the active ingredient(s) that is effective to achieve the desired therapeutic response for a particular subject.

For any API of the disclosure, duration of treatment could range from a single dose administered on a one-time basis to a life-long course of therapeutic treatments. The duration of treatment can and will vary depending on the subject, the API, and the condition to be treated. For example, the duration of treatment may be 1 day, 2 days, 3 days, 4 days, 5 days, 6 days, or 7 days. Or, the duration of treatment may be for 1 week, 2 weeks, 3 weeks, 4 weeks, 5 weeks, or 6 weeks. Alternatively, the duration of treatment may be for 1 month, 2 months, 3 months, 4 months, 5 months, 6 months, 7 months, 8 months, 9 months, 10 months, 11 months, or 12 months. The duration of treatment may also be for 1 year, 2 years, 3 years, 4 years, 5 years, or greater than 5 years. It is also contemplated that administration may be frequent for a period of time, and then administration may be spaced out for a period of time. For example, duration of treatment may be 5 days, then no treatment for 9 days, then treatment for 5 days.

In some aspects, a composition of the disclosure is administered as needed, upon development or shortly before development of symptoms. For instance, when the API is sumatriptan, the composition is administered upon development of migraine, or shortly before development of the migraine, when symptoms associated with impending migraines appear.

The timing of administration of the treatment relative to the condition to be treated itself and duration of treatment will be determined by the circumstances surrounding the case. Treatment may begin immediately, such as at the time of diagnosis, or treatment could begin following other therapies. Treatment may begin in a hospital or clinic itself, or at a later time after discharge from the hospital or after being seen in an outpatient clinic.

Administration of the compositions described herein may be carried out as part of a treatment regimen that may include multiple instances of administration of one or more compositions comprising the API, as well as administration of other pharmaceutically active compositions. Such a regimen may be designed as a method of immediately treating a condition such as, for immediate relief of migraine pain, and/or as a method of long-term maintenance of the health of a subject after having been treated for a condition (e.g., prevention). A treatment regimen may be designed as a method of treating an asymptomatic subject. Such treatment regimen may delay the onset of the condition of interest in a subject. It will be appreciated that determination of appropriate treatment regimens is within the skill of practitioners in the art.

It will be appreciated by those skilled in the art that a combination of more than one composition of the present disclosure may be used. It will also be appreciated by those skilled in the art that a composition of the present disclosure may be used in combination with other therapeutic agents before, after, and/or during treatment with a composition of the disclosure. Further, methods of the invention may be used in combination with standard treatments for the specific lysosomal storage disorder and disorders characterized by lysosomal dysfunction.

A selected dosage level may depend upon a variety of factors, including the specific API in a composition, the activity of the therapeutic composition, formulation, the route of administration, combination with other drugs or treatments, disease and longevity, and the physical condition and prior medical history of the subject being treated. For instance, when the active ingredient is sumatriptan, the presence of sumatriptan in the composition and the intended route of administration of the composition comprising sumatriptan may factor into the selected dosage level of sumatriptan.

When sumatriptan is administered nasally, a composition comprising sumatriptan may be administered at a dose ranging from about 1 to about 15 mg/Kg body weight, from about 5 to about 12 mg/Kg body weight, or from about 8 to about 11 mg/Kg body weight. When a composition comprising sumatriptan is administered at a dose ranging from about 8 to about 11 mg/Kg body weight, administering a single dose can provide a Cₘₐₓ serum level of sumatriptan ranging from about 1000 ng/ml to about 3000 ng/ml, or about 1200 ng/ml to about 2000 ng/ml at a Tₘₐₓ ranging from about 0.5 seconds to about 10 minutes, or about 1 second to about 5 minutes.

### III. Kits

A further aspect of the present disclosure provides a pharmaceutical kit comprising a pharmaceutical composition detailed above in Section I for transmucosal administration of an active pharmaceutical ingredient (API). The pharmaceutical kit further comprises an applicator for use in facilitating the transmucosal administering of the API. The applicator can be a metered dose dispenser, a non-metered dose dispenser, a squeezable dispenser, a pump dispenser, a spray dispenser, a foam dispenser, an aerosol dispenser, a dispenser containing a propellant, an inhalation dispenser, a soft pipette with an elastomeric bulb in fluid communication with a reservoir containing the composition, a dropper for directing into the nose, a swab having an absorbent portion impregnated with the composition, a swab having an anatomically-shaped portion comprising an absorbent portion impregnated with the composition, and a swab having a compressed absorbent portion in fluid communication with a reservoir containing the composition.

The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The term “a therapeutically effective amount” as used herein refers to an amount effective, at dosages, and for periods of time necessary, to achieve the desired result with respect to the treatment of a disease. For example, in the treatment of a cancer, an agent (i.e., a compound or a composition) which decreases, prevents, delays or suppresses or arrests any symptoms of the cancer would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period. A therapeutically effective amount may be determined by the efficacy or potency of the particular composition, the disorder being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining therapeutically effective amounts, one skilled in the art may also consider the existence, nature, and extent of any adverse effects that accompany the administration of a particular compound in a particular subject.

As used herein, the terms “treating,” “treatment,” or “to treat” each may mean to alleviate, suppress, repress, eliminate, prevent or slow the appearance of symptoms, clinical signs, or underlying pathology of a condition or disorder on a temporary or permanent basis. Preventing a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment may reduce the risk of developing the condition and/or lessen its severity if the condition later develops. For instance, treatment of a microbial infection may reduce, ameliorate, or altogether eliminate the infection, or prevent it from worsening.

The term “transmucosal administration” relates to a delivery system through mucosa, the mucosa may be of oral, buccal, sublingual, gingival, palatal, nasal, nasopharynxal, oropharynxal, conjunctival, vaginal, and gastrointestinal membranes in which a substantial portion of the active agent enters the blood stream of the subject by diffusion or movement through the mucosal membranes.

The terms “intranasal administration” or “nasal administration” are used interchangeably, and relate to a delivery system through the nasal mucosa in which a substantial portion of the active agent enters the blood stream of the subject by diffusion or movement through the nasal mucus membrane.

As used herein “peak concentration (Cₘₐₓ)”, “area under concentration vs. time curve (AUC)”, “time to maximal plasma concentration (tₘₐₓ)” of triptans in blood (or CSF) are pharmacokinetic parameters known to those skilled in the art. The “concentration vs. time curve”, also termed the pK curve, measures the concentration of an API in a blood serum of a subject over time after administration of a dosage of an API to the subject. “Cₘₐₓ” is the mean maximum concentration of therapeutic achieved, usually in blood, following a single dosage of a drug to an experimental population of mammals or test subjects. “Tₘₐₓ” is the mean time to reach maximum concentration of an API, usually for an experimental population of animals or test subjects, following administration of a single dosage of drug to each subject.

The term “Mucosal permeation enhancer” denotes any penetration enhancer that assists in penetration of the API via mucosal membrane. The term “nasal permeation enhancer” denotes any penetration enhancer that assists in penetration of the API via the nasal mucosal membrane. It includes, but is not limited to, BZK and PG.

The term “about” denotes a range of value ranging from −10% of the value it modifies to +10% of the value it modifies, with the proviso that such claim construction does not preclude application of broader equivalent values.

As various changes could be made in the above-described compositions and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Effect of Propylene Glycol and Benzalkonium Chloride on the Pharmacokinetic Profile of Sumatriptan

The pharmacokinetic profiles of 4 different formulations of Sumatriptan were compared. The formulations are as described in Table 1.

TABLE 1
Formulation details.

| | Ingredients | S07D038 | S07D039 | S07D040 | 123669 |
|---|---|---|---|---|---|
| 1 | potassium phosphate | 0.85 | 0.85 | — | 1.3 |
| | monobasic | | | | |
| 2 | sodium phosphate | 0.1 | 0.1 | — | 0.2 |
| | dibasic anhydrous | | | | |
| 3 | Sumatriptan | 9.52 | 9.52 | 9.52 | 18.52 |
| 4 | Citric acid | 2.81 | 2.82 | 2.86 | — |
| | monohydrate | | | | |
| 5 | Benzalkonium chloride | 0.02 | 0.119 | 0.119 | 0.02 |
| 6 | Propylene glycol | — | — | 3.5 | — |
| 7 | Edetate disodium | — | — | — | 0.01 |
| 8 | Sodium hydroxide | — | — | — | 0.373 |
| 9 | Sulfuric acid | — | — | — | 3.5 |
| 10 | Purified Water | Q.S. | Q.S. | Q.S. | Q.S. |

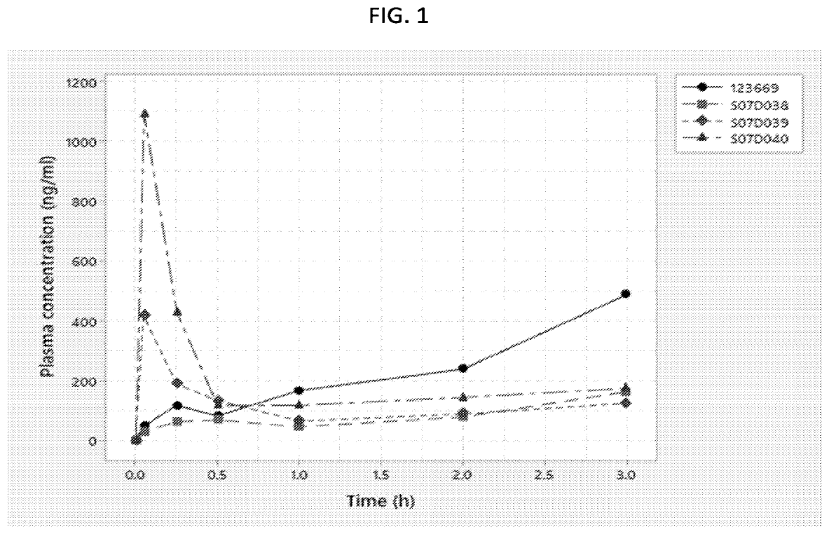

The results of pharmacokinetic analysis of each formulation are shown in Table 2 and FIG. 1.

TABLE 2
Calculated pharmacokinetic parameters.

| Tested | | | | |
|---|---|---|---|---|
| pharmacokinetic | | | | |
| parameters | S07D038 | S07D039 | S07D040 | 123669 |
| Dose (mg/animal) | 3 | 3 | 3 | 6 |
| Dose (mg/Kg) | 9.3 | 9.2 | 9.3 | 18.7 |
| Cₘₐₓ (ng/ml) | 163.38 | 417.8 | 1090.77 | 487.16 |
| Tₘₐₓ (h) | 3 | 0.05 | 0.05 | 3 |
| AUC_{0-3 h} (ng/ml*h) | 241.8 | 347.7 | 593.3 | 629.8 |

The leading steps in preparing the formulations are:
In a mixer container, dissolve dibasic sodium phosphate, anhydrous and monobasic potassium phosphate, in purified water.
Once a clear solution is obtained, add Sumatriptan and mix.
In a separate vessel, dissolve citric acid monohydrate, in purified water.
Once a clear solution is obtained, add to the mixer and mix until a clear solution is obtained.
In a separate vessel dissolve BZK in purified water.
Once a clear solution is obtained, add to the mixer and mix until a clear solution is obtained. Add PG to the mixer and mix.

Five groups of 12 male Sprague Dawley SD rats were each administrated the 5 different formulations of Sumatriptan. The formulations were administered by intranasal instillation of 15 μL in each nostril. The dose was administered to each animal by means of a graduated microliter syringe. All animals were dosed once only.

Blood samples were collected at 8 time points for each group, at the following time points: pre-dose, 3, 15, 30, 60 minutes, and 2, 3, and 4 hours after dosing. At each sampling time, approximately 0.5 m L blood samples were withdrawn from the tail vein of 3 males of each group, each animal being sampled at 2 alternating time points.

Samples were transferred into tubes containing K₃EDTA as anticoagulant and centrifuged at room temperature. The plasma was divided into a first aliquot of at least 100 μL, and a second aliquot of the remainder amount of plasma, and frozen at 18° C. until analysis for Sumatriptan concentrations.

Serum levels of Sumatriptan are shown in FIG. 1.

The results show that:
1. PG and higher concentration of BZK have a significant impact on the pharmacokinetic profile of Sumatriptan by enabling rapid penetration of the drug.
2. Faster Tₘₐₓ and higher Cₘₐₓ and AUC were achieved at a concentration of 0.119% w/w BZK when comparing to a lower concentration of 0.02% w/w.
3. Higher Cₘₐₓ was achieved at a concentration of 0.119% and 3.5% PG than by BZK alone (Tₘₐₓ remained the same).

Table 2 and FIG. 1 demonstrate that the combination of PG and higher concentration of BZK has a significant impact on the pharmacokinetic profile of the API. Formulations containing higher concentration of BZK reduce the Tₘₐₓ compared to formulations containing lower concentration of BZK. Formulations containing higher concentration of BZK have higher Cₘₐₓ compared to formulations with lower concentration of BZK. The combination of PG and BZK increases the Cₘₐₓ of the API compared to the formulation containing BZK alone.

### Example 2. Effect of Different Concentrations of Propylene Glycol on Pharmacokinetic Profile of Sumatriptan

The results in Example 1 show that 3.5% of PG has a significant impact on the pharmacokinetic profile. This study was conducted to determine the impact of the concentrations of 1.5% and 2.5% PG.

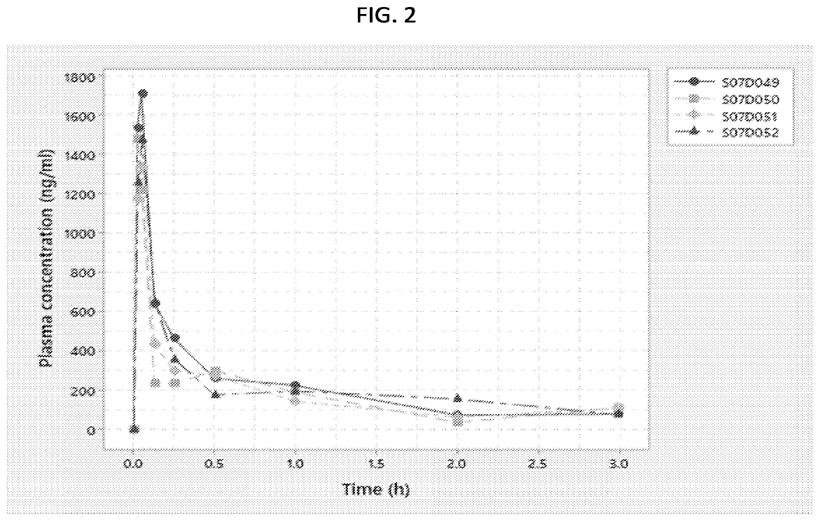

The experimental design and methods were as described in Example 1. The formulations tested and calculated pharmacokinetic parameters are shown in Tables 3-4 below and FIG. 2.

TABLE 3
Formulation details

| | Ingredients | S07D049 | S07D050 | S07D051 | S07D052 |
|---|---|---|---|---|---|
| 1 | potassium phosphate | — | 0.0425 | 0.0425 | 0.0425 |
| | monobasic | | | | |
| 2 | sodium phosphate | — | 0.005 | 0.005 | 0.005 |
| | dibasic anhydrous | | | | |
| 3 | Sumatriptan | 9.52 | 9.52 | 9.52 | 9.52 |
| 4 | Citric acid | 2.92 | 2.84 | 2.85 | 2.84 |
| | monohydrate | | | | |
| 5 | Benzalkonium | 0.119 | 0.119 | 0.119 | 0.119 |
| | chloride | | | | |
| 6 | Propylene glycol | 3.5 | 3.5 | 2.5 | 1.5 |
| 7 | Purified Water | Q.S. | Q.S. | Q.S. | Q.S. |
| | | 100% | 100% | 100% | 100% |

* S07D049 is identical to S07D040

TABLE 4
Calculated pharmacokinetic parameters

| Tested | | | | |
|---|---|---|---|---|
| pharmacokinetic | | | | |
| parameters | S07D049 | S07D050 | S07D051 | S07D052 |
| Dose (mg/animal) | 3 | 3 | 3 | 3 |
| Dose (mg/Kg) | 9.7 | 9.6 | 9.7 | 9.8 |
| Cₘₐₓ (ng/ml) | 1708.7 | 1478.9 | 1335.5 | 1470.0 |
| Tₘₐₓ (h) | 0.05 | 0.025 | 0.05 | 0.05 |
| AUC_{0-Tlast} (ng/ml*h) | 654.7 | 511.4 | 522.6 | 634.7 |

In this experiment three formulations with different PG concentrations were tested: 3.5% w/w (batch #S07D049; batch #S07D050), 2.5% w/w (batch #S07D051) and 1.5% (batch #S07D052). The formulations were introduced to the rats as detailed above in Example 1, and plasma concentration of the API was tested. The results of the pharmacokinetic analysis shown in table 4 and FIG. 2 show that there was no significant difference in Cₘₐₓ and Tₘₐₓ between the formulations containing concentration of PG. The Tₘₐₓ of the formulations in this study was similar to the Tₘₐₓ of the formulations in Example 1 in formulations that contained the same concentration of BZK.

1. A pharmaceutical composition for intranasal administration of an active pharmaceutical ingredient (API), comprising:
a. the API; and
b. penetration enhancing agents, the agents comprising:
i. benzalkonium chloride; and
ii. propylene glycol.

2. The composition of claim 1, wherein intranasal administration is administered as a spray, drops, an aerosol, a paste, a cream, a gel, an ointment, a salve, a foam, a lotion, an oil suspension, an emulsion, or a solution.

3. The composition of claim 1, wherein the penetration enhancing agents comprise benzalkonium chloride at a concentration ranging from about 0.05% to about 0.5% w/w.

4. The composition of claim 1, wherein the penetration enhancing agents comprise propylene glycol at a concentration ranging from about 1.5% to about 12% w/w.

5. The composition of claim 1, wherein the penetration enhancing agents comprise benzalkonium chloride at a concentration ranging from about 0.05% to about 0.5% w/w, and propylene glycol at a concentration ranging from about 1.5% to about 12% w/w.

6. The composition of claim 1, further comprising a pH modifier.

7. The composition of claim 1, wherein the API is a painkiller.

8. The composition of claim 7, wherein the painkiller is a migraine medication.

9. The composition of claim 1, wherein the API is Naloxone, zolmitriptan, midazolam, diazepam, epinephrine, esketamin, painkillers, sumatriptan, nitroglycerin, or combinations thereof.

10. The composition of claim 1, wherein the API is sumatriptan.

11. The composition of claim 10, wherein the sumatriptan is at a concentration ranging from about 9% to about 11% w/w of the composition.

12. The composition of claim 10, wherein administration of the composition provides a Cₘₐₓ ranging from about 1.5 times to about 3 times higher than the Cₘₐₓ obtained upon administration of a composition comprising sumatriptan and BZK without PG.

13. A method of intranasal administration of a pharmaceutically active ingredient (API) to a subject in need thereof, the method comprising administering through an intranasal route of administration to the subject the composition of claim 1.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A pharmaceutical composition for intranasal administration of sumatriptan, comprising:
a. about 9% to about 11% w/w sumatriptan; and
b. penetration enhancing agents, the agents comprising:
i. about 0.05% to about 0.5% w/w benzalkonium chloride; and
ii. about 1.5% to about 12% w/w propylene glycol.

27. A method of intranasal administration of sumatriptan to a subject in need thereof, the method comprising administering to the subject through an intranasal route of administration a composition
ii. of claim 26.

28. A method of treating pain in a subject in need thereof, the method comprising intranasal administering to the subject a composition comprising:
a. a painkiller; and
b. penetration enhancing agents, the agents comprising:
i. benzalkonium chloride; and
ii. propylene glycol.

29. The method of claim 28, wherein the penetration enhancing agents comprise benzalkonium chloride at a concentration ranging from about 0.05% to about 0.5% w/w.

30. The method of claim 28, wherein the penetration enhancing agents comprise propylene glycol at a concentration ranging from about 1.5 to about 12% w/w.

31. The method of claim 28, wherein the penetration enhancing agents comprise benzalkonium chloride at a concentration ranging from about 0.05% to about 0.5% w/w, and propylene glycol at a concentration ranging from about 1.5% to about 12% w/w.

32. The composition of claim 28, wherein the pain is migraine pain.

33. The composition of claim 28, wherein the painkiller is a migraine medication.

34. The method of claim 28, wherein the painkiller is sumatriptan.

35. The method of claim 34, wherein the sumatriptan is at a concentration ranging from about 9% to about 11% w/w of the composition.

36. The method of claim 34, wherein the penetration enhancing agents comprise benzalkonium chloride at a concentration ranging from about 0.05% to about 0.5% w/w and propylene glycol at a concentration ranging from about 1.5% to about 12% w/w.

37. The method of claim 34, wherein administration of the composition provides a Cₘₐₓ ranging from about 1.5 times to about 3 times higher than the Cₘₐₓ obtained upon administration of a composition comprising sumatriptan and BZK without PG.

38. A method of treating pain in a subject in need thereof, the method comprising intranasal administering to the subject the composition
ii. of claim 28.

39. The method of claim 38, wherein the painkiller is sumatriptan.

40. The method of claim 39, wherein the sumatriptan is at a concentration ranging from about 9 to about 11% w/w of the composition.

41. A method of treating a migraine in a subject in need thereof, the method comprising intranasally administering a composition comprising a migraine medication to the subject, the composition comprising:
a. a migraine medication; and
b. penetration enhancing agents, the agents comprising:
i. benzalkonium chloride; and
ii. propylene glycol.

42. (canceled)

43. (canceled)

44. (canceled)

45. (canceled)

46. (canceled)

47. (canceled)

48. (canceled)

49. A method of treating migraine pain in a subject in need thereof, the method comprising intranasally administering to the subject the composition
ii. of claim 26.

50. A pharmaceutical kit comprising the pharmaceutical composition of claim 1 for intranasal administration of an active pharmaceutical ingredient (API).
